(19) United States
(12) Patent Application Publication
(10) Pub. No.: US 2025/0009938 A1
(43) Pub. Date: Jan. 9, 2025

(54) CELLULARIZED NERVE REGENERATION GRAFT AND METHODS OF MAKING THE SAME

(71) Applicant: Rutgers, The State University of New Jersey, New Brunswick, NJ (US)

(72) Inventors: Zhiping Pang, New Providence, NJ (US); N. Sanjeeva Murthy, Princeton, NJ (US); Andrew J. Boreland, Annandale, NJ (US); Jasmine M. Gamboa, Union City, NJ (US); Jeremy M. Perrelle, New Brunswick, NJ (US)

(73) Assignee: Rutgers, The State University of New Jersey, New Brunswick, NJ (US)

(21) Appl. No.: 18/696,883

(22) PCT Filed: Sep. 28, 2022
(86) PCT No.: PCT/US2022/077140

§ 371 Date: Mar. 28, 2024

Related U.S. Application Data
(60) Provisional application No. 63/249,353, filed on Sep. 28, 2021.

(51) Int. Cl. A61L 27/38; A61L 27/36; A61L 27/52; A61L 27/58; C12N 5/077; C12N 5/079

(52) U.S. Cl. CPC A61L 27/3891; A61L 27/3675; A61L 27/3804; A61L 27/383; A61L 27/52; A61L 27/58; C12N 5/0622; C12N 5/0656; A61L 2430/32; C12N 2537/00

(57) ABSTRACT

A cellularized nerve regeneration graft is disclosed that includes an electrospun biodegradable polymer conduit having an exterior surface and an interior luminal space, a plurality of fibroblasts seeded to the exterior surface of the conduit, and a system filling the interior luminal space of the conduit. The system may include a hydrogel matrix or augmented hydrogel matrix and Schwann cells. The cellularized nerve regeneration graft may be used in the repair of peripheral nerve injuries. Methods of making the cellularized nerve regeneration graft are also disclosed.

### FIELD OF THE DISCLOSURE

This disclosure is related to a cellularized nerve regeneration graft for use in repairing peripheral nerve injuries, and methods of making the same.

### BACKGROUND OF THE DISCLOSURE

Peripheral Nerve Injuries (PNI) cost the global economy over $150 billion USD annually in direct medical expenditures. In addition, there are other costs including lost wages, indirect expended and other long-term social and economic costs of loss-of-function PNI for individuals and their communities, particularly since the median age of such injuries is approximately 35 years. Such injuries can arise from physical trauma, cancers, or other nervous system pathologies. Associated socioeconomic costs multiply considerably in a global context, particularly in developing countries where jobs involving manual labor lead to more frequent workplace injuries. Despite major advances in medical care and increased neurosurgical precision, nerve injuries still lack much hope for complete functional recovery, often leaving patients without sensation and/or motor function. Furthermore, understanding of nerve pathologies in both the PNS and CNS disorders is in its infancy with few solutions to degenerative nerve diseases. Prior experiments have demonstrated the efficacy of generating multi-layer biopolymer scaffolds to direct growth of support cells into a matrix that mimics the human extracellular matrix. See, e.g., Garrison, CM, Singh-Varma, A, Pastino, A K, et al., “A multilayered scaffold for regeneration of smooth muscle and connective tissue layers,” J Biomed Mater Res. 2020; 109:733-744. Current understanding of tissue development suggests that the ECM, support cells, and regulatory molecules such as growth factors play integral roles in tissue growth and remodeling.

Though surgical techniques to repair nerve injuries have improved, there is still no “gold standard” treatment for nerve damage. According to Dr. Susan E. Mackinnon, a pioneer in nerve graft surgery, the best nerve graft—the autologous graft—is considered a mere “bronze standard.” Current tissue grafts return most patients only to partial functional and sensory capacity after surgery. Additionally, the cost of managing PNIs is on the rise. Currently, repair of nerve injuries costs approximately $47,000 per patient at the time of repair, with an annual increase of 9.69%. In addition, the understanding of neuron regeneration and alleviation of debilitating nervous system conditions is still in its infancy.

### SUMMARY

A cellularized nerve regeneration graft is disclosed comprising: an electrospun biodegradable polymer conduit having an exterior surface and an interior luminal space; a plurality of fibroblasts seeded to the exterior surface of the conduit; and a system comprising a hydrogel matrix or augmented hydrogel matrix and Schwann cells, wherein the system fills the interior luminal space of the conduit. The augmented hydrogel matrix may comprise a hydrogel selected from the group consisting of: RADA-16 peptides, collagen, gelatin, alginate, hyaluronic acid, or any combination thereof, combined with a growth factor, guidance cue, structural peptide, adhesion factor, other chemical agent, or any combination thereof. The electrospun biodegradable polymer conduit may comprise a plurality of layers, for example, two layers with one layer having aligned biopolymer fibers and the second layer having unaligned biopolymer fibers. The electrospun biodegradable polymer may be a tyrosine-derived or tyrosol-derived polymer. The fibroblasts may form a continuous cell layer on the exterior surface of the conduit at a concentration of greater than about 1.0×10⁵ cells/mL/cm². The Schwann cells may be substantially evenly distributed throughout the hydrogel matrix or augmented hydrogel matrix within the interior luminal space of the conduit, for example, at a concentration of about greater than 20 million cells/mL. The cellularized nerve regeneration graft may comprise a plurality of channels running in a longitudinal direction of the electrospun biodegradable polymer conduit and within the system. The plurality of channels may be hollow and reinforced with Schwann cells.

A method of making the cellularized nerve regeneration graft is also disclosed. The method includes electrospinning a formulation of a tyrosine-derived or tyrosol-derived polymer to make the electrospun biodegradable polymer conduit; culturing fibroblasts; seeding the fibroblasts on the exterior surface of the electrospun biodegradable polymer conduit; generating human Schwann cells; embedding the human Schwann cells in the hydrogel matrix or augmented hydrogel matrix to make the system; seeding the system in the interior luminal space of the electrospun biodegradable polymer conduit; and incubating the seeded conduit to make the cellularized nerve regeneration graft.

Also disclosed is a method of repairing an injured peripheral nerve in a patient comprising implanting the cellularized nerve regeneration graft into the patient.

A method of making the cellularized nerve regeneration graft having a plurality of channels running in a longitudinal direction of the electrospun biodegradable polymer conduit is disclosed. That method may include: electrospinning a formulation of tyrosine-derived or tyrosol-derived polymer to make the electrospun biodegradable polymer conduit; generating human Schwann cells; embedding the human Schwann cells in the hydrogel matrix or augmented hydrogel matrix to make the system; either culturing resorbable fibers with the human Schwann cells and then loading the cultured resorbable fibers into the electrospun biodegradable polymer conduit in a longitudinal arrangement, or loading the resorbable fibers into the electrospun biodegradable polymer conduit in a longitudinal arrangement, followed by adding a suspension comprising human Schwann cells into the interior luminal space of the electrospun biodegradable polymer conduit to make cultured resorbable fibers; seeding the system into the interior luminal space of the electrospun biodegradable polymer conduit and between the cultured resorbable fibers; and incubating the seeded conduit for about 1 to about 6 weeks, wherein the resorbable fibers dissolve leaving the cellularized nerve regeneration graft having a plurality of longitudinal hollow channels therein, optionally having Schwann cells within the channels. The method may further comprise: culturing fibroblasts; and seeding the fibroblasts on the exterior surface of the electrospun biodegradable polymer conduit. The resorbable fibers may be made from poly(lactic-co-glycolic acid) (PLGA), polyglycolic acid (PGA), a tyrosine-derived polymer with a high concentration of poly(ethylene glycol) (PEG), or any combination thereof. The spacing between the cultured resorbable fibers may be about 20 μm to about 100 μm.

### BRIEF DESCRIPTION OF THE FIGURES

FIG. 1A-B are electron micrograph images showing aligned and unaligned fibers in a bilayered sheet.

FIG. 2A-B are electron micrograph images showing aligned and unaligned fibers in a bilayered sheet.

FIG. 3A-E is an example of the electrospun biodegradable polymer conduit having the system of augmented hydrogel matrix and embedded Schwann cells enclosed within its inner luminal space.

FIG. 4A-F is an example of various 3-D printed parts used to assemble a cellularized nerve regeneration graft of the disclosure.

FIG. 5 is a schematic demonstrating an example of how Schwann cells may be cultured onto resorbable fibers to make longitudinal channels within a polymer conduit.

FIG. 6 is a schematic for making a cellularized nerve regeneration graft of the disclosure.

FIG. 7A-D is an example of the construction of a multi-layer biopolymer fixture for initial cell culture.

FIG. 8A-E is an example of the construction of a tyrosine-derived biodegradable polymer conduit and fixture assembly.

FIG. 9A-C is an example of a method of generating human Schwann cells, and graph of the Schwann Cell fold expression.

FIG. 10 depicts a second method of generating human Schwann cells using transcription factors delivered by lentivirus in combination with growth factors to drive differentiation of either iPSCs or fibroblasts (FBs) to Schwann cells.

FIG. 11A-C is a representation of Schwann cells and fibroblasts cultured onto opposite sides of an electrospun biodegradable polymer fiber scaffold.

FIG. 12 is a scheme for implantation of a cellularized nerve regeneration graft of the disclosure into a murine peripheral nerve injury model.

FIG. 13 is an example of magnification imaging of rat Schwann cells growing on fibers made from a tyrosine-derived polycarbonate.

FIG. 14A-C are cross-sectional confocal microscopy images demonstrating rat Schwann cells proliferating within collagen hydrogel matrix; tyrosine-derived polymer fibers are shown dispersed throughout the matrix, maintaining longitudinal tracts in the hydrogel matrix.

### DETAILED DESCRIPTION

A method is disclosed for generating a biodegradable polymer conduit seeded with neuronal support cells to facilitate axonal regeneration in PNI patients. This includes construction of a cellularized nerve regeneration graft (CNRG) that is suitable for connecting the injured peripheral nerve tissue. Stem cell biology, biomaterial and 3D printing are combined to create the CNRG.

The cellularized nerve regeneration graft disclosed herein utilizes an optionally multi-layered electrospun biodegradable polymer conduit seeded with fibroblasts (FBs) in its exterior surface and Schwann cells (SCs) in its interior luminal space. These Schwann cells may be derived through an induced pluripotent stem cell pathway to minimize graft rejection and facilitate axonal regrowth into a favorable regenerative environment.

A cellularized nerve regeneration graft is disclosed comprising: an electrospun biodegradable polymer conduit having an exterior surface and an interior luminal space; a plurality of fibroblasts seeded to the exterior surface of the conduit; and a system filling the interior luminal space of the conduit. The system comprises a hydrogel matrix or augmented hydrogel matrix and Schwann cells. The cellularized nerve regeneration graft may be a solid structure containing Schwann cells throughout the interior luminal space, with fibroblasts localized on the external surface. The Schwann cells may grow 3-dimensionally throughout the hydrogel matrix or augmented hydrogel matrix.

A patient may be prepared for surgery and the cellularized nerve regeneration graft, containing live cells and growth factors, implanted into the patient by suturing or otherwise adhering the terminal ends of the graft to the distal and proximal ends of the nerve injury.

Thus, a pre-established cellular structure is developed prior to implantation, such that a pre-established tissue will promote cell survival once implanted and will constitute a “true-to-in vivo” environment upon implantation. Previous approaches have simply injected Schwann cells into a gel without growing them to a stable culture within the hydrogel prior to implantation. By developing a pre-established tissue-like construct, this will mitigate post-implantation toxic apoptotic byproducts and reinforce the authentic cell-cell interactions present in living tissue. The role of fibroblasts is to establish the basal directionality for Schwann cells and to provide growth and adhesion factors for the Schwann cells, thus mimicking authentic nerve architecture.

The electrospun biodegradable polymer conduit may have a diameter of about 1.0 mm to about 5.0 mm, or about 1.5 mm to about 4.0 mm. The conduit may have a length of about 1.0 cm to about 10.0 cm, or about 1.0 cm to about 5.0 cm. The conduit may have a thickness of about 50 μm to about 500 μm, or about 50 μm to about 300 μm. The conduit may be constructed from a tyrosine-derived or tyrosol-derived polymer, which may alternatively be referred to as a tyrosine-polymer, or tyrosol-derived polymer, respectively. The tyrosine-derived or tyrosol-derived polymer have non-inflammatory degradation bioproducts. The conduit may be composed of a tyrosine-derived polymer, for example, desaminotyrosyl-tyrosine ethyl ester (DTE), desaminotyrosyl-tyrosine (DT), or a combination thereof. The conduit may be composed of desaminotyrosyl-tyrosine ethyl ester (DTE), desaminotyrosyl-tyrosine (DT), and polyethylene glycol (PEG). The molar fraction of free carboxylic acid units and PEG units in the polymer described herein can be adjusted to modify the mechanical properties and degradation rates of NADs made from such polymers. For example, polymers with lower amounts of free carboxylic acid will tend to have longer lifetimes in the body. Further, by otherwise adjusting the amount of free carboxylic acid in the polymers across the range of preferred molar fraction, the resulting polymers can be adapted for use in various applications requiring different device lifetimes. In general, the higher the molar fraction of free carboxylic acid units, the shorter the lifetime of the device in the body and more suitable such devices are for applications wherein shorter lifetimes are desirable or required.

The conduit may be composed of a tyrosol-derived polymer, for example, U.S. Publication No. 2020/0181321 and WO 2021/055090, which are incorporated by reference herein in their entirety. The conduit may be composed of poly(HTy glutarate), poly(HTy suberate), poly(HTY dodecanedioate), poly(HTy phenylenediacetate), or any combination thereof. The conduit may be composed of poly(HTy glutarate).

The electrospun biodegradable polymer conduit may be constructed from a biodegradable polymer having repeating units of the structure (Formula I):

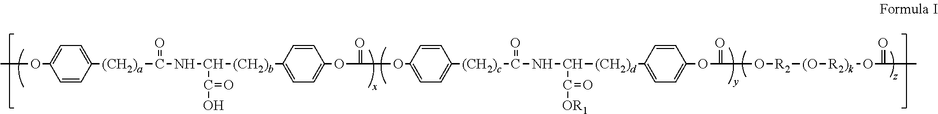

wherein a and b are independently 0 or an integer between 1 and 6, inclusive; wherein c and d are independently 0 or an integer between 1 and 6, inclusive; wherein each R₁ is independently selected from the group consisting of straight and branched alkyl groups containing up to 18 carbon atoms; wherein each R₂ is independently an alkylene group containing up to 6 carbon atoms; wherein k is between about 20 and about 200; and wherein x ranges between about 0.02 and about 0.20; z ranges between about 0.005 and 0.10; and x+y+z=1.00.

In some embodiments, a and b are two and one, respectively. In some embodiments, c and d are two and one, respectively, and R₁ is ethyl. In some embodiments, R₂ for said polymer is ethylene and k is between about 25 and about 50.

The synthesis of various polycarbonate polymers is generally known in the art, including for example, the methods disclosed in U.S. Pat. Nos. 6,120,491 and 6,475,477, the disclosures of which are incorporated herein by reference. The polyacrylates and other polycarbonates disclosed in U.S. Pat. No. 6,120,491 are also incorporated herein by reference for use in construction of the polymer conduits herein. Polymers having pendent free carboxylic acid groups are preferably prepared from the corresponding benzyl and tert-butyl ester polymers to avoid cross-reaction of the free carboxylic acid group with co-monomers. The benzyl ester polymers may be converted to the corresponding free carboxylic acid polymers by the palladium catalyzed hydrogenolysis method disclosed in U.S. Pat. No. 6,120,491. The tert-butyl ester polymers may be converted to the corresponding free carboxylic acid polymers through the selective removal of the tert-butyl groups by the acidolysis method disclosed in U.S. Patent Publication No. 20060034769, also incorporated herein by reference.

Polymers may be selected which degrade or resorb within a predetermined time. For this reason, embodiments may include polymers with molar fractions of monomeric repeating units with pendant fee carboxylic acid groups, such as DT, between about 2 and about 20 mol %, and preferably between about 5 and about 20 mol %.

Poly(alkylene glycol) segments, such as PEG, decrease the surface adhesion of the polymers. By varying the molar fraction of poly(alkylene glycol) segments in the block copolymers provided by the present invention, the hydrophilic/hydrophobic ratios of the polymers can be changed to adjust the ability of the polymer coatings to modify cellular behavior. Increasing levels of poly(alkylene glycol) inhibit cellular attachment, migration and proliferation. Secondarily, PEG increases the water uptake, and thus increases the rate of degradation of the polymer. Accordingly, in an embodiment, polymers are selected in which the amount of poly(alkylene glycol) is limited to between 0.5 and about 10 mol %, and preferably between about 0.5 and about 5 mol %, and more preferably between about 0.5 and about 1 mol %. The poly(alkylene glycol) may have a molecular weight of 1 k to 2 k.

The polymer may be selected having intrinsic physical properties appropriate for use in polymer conduits with suitable mechanical properties including elasticity, rigidity, strength and degradation behavior. Such polymers include, if the polymer is amorphous, polymers with a glass transition temperature greater than 37° C. when fully hydrated under physiological conditions and, if the polymer is crystalline, a crystalline melting temperature greater than 37° C. when fully hydrated under physiological conditions.

It is to be understood that other biodegradable and biocompatible polymers can be used to form fibers that provide or reinforce certain desirable properties of the resulting polymer conduits. Examples of other polymers that may be used include, but are not limited to, poly(lactic acid), poly(glycolic acid), poly(lactic-co-glycolic acid), polycaprolactone, various poly(amino acid) s and polyanhydrides. Other natural or non-natural fiber materials, for example, collagen, cellulose, chitosan, and their derivatives, may alternatively or additionally be utilized to provide or reinforce certain desirable properties of the resulting polymer conduits (see, for example, U.S. Pat. No. 8,216,602).

The electrospun biodegradable polymer conduit may be constructed from any polymer disclosed in U.S. Patent Publication No. 2018/0280567, which is incorporated by reference herein in its entirety.

The electrospun biodegradable polymer conduit may be constructed from a biocompatible polymer comprising a recurring unit of Formula XVIII:

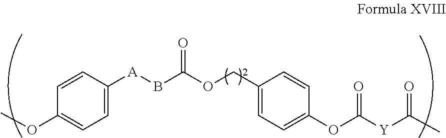

wherein:
(a) A is CH₂ or CH₂CH₂, B is a bond, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀;
or
(b) A is CH₂CH₂, B is selected from the group consisting of —O—CO—CH₂CH₂, —O—CO—CH₂CH₂CH₂, and —O—CO—CH₂OCH₂ and bonded to A via oxygen, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀, or any other polymer disclosed in U.S. Patent Publication No. 2020/0181321, which is incorporated by reference herein in its entirety. The electrospun biodegradable polymer conduit may be constructed from PEG block polymers of the foregoing polymer.

The electrospun biodegradable polymer conduit may be constructed from a biocompatible polymer comprising a plurality of units of Formula I,

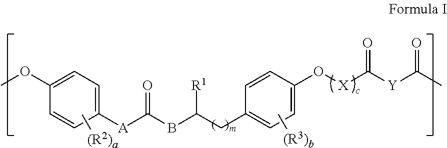

wherein:

A is a bond, C₁₋₃alkylene, OC₁₋₃alkylene, CH═CH, C₁₋₃alkylene-O—CO—C₂₋₅alkylene, C₁₋₃alkylene-O—CO—C₁₋₂alkylene-O—C₁₋₂alkylene;

B is oxygen, or NR^{a} wherein R^{a} is H or an optionally substituted C₁₋₁₀ alkyl, or —O-E-OCO— wherein E is selected from the group consisting of C₁₋₃₀ alkylene, C₂₋₃₀ alkelene, C₁₋₃₀ alkynylene, C₁₋₃₀ heteroalkylene, C₂₋₃₀ heteroalkelene, C₁₋₃₀ heteroalkynylene, C₆₋₃₀ arylene, C₇₋₃₀ alkylarylene, C₈₋₃₀ alkenlarylene, C₈₋₃₀ alkynylarylene, and C₂₋₃₀ heteroarylene,

R¹ is H or COORD wherein Rb is selected from the group consisting of H, C₁₋₁₀ alkyl and C₁₋₁₀ alkylaryl containing up to 18 carbon atoms;
R² and R³ are independently halogen, C₁₋₄alkyl or OC₁₋₄alkyl;
X in each instance is an amino acid moiety represented as

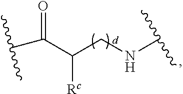

wherein
R^{c} is H, or an unsubstituted or substituted group selected from the group consisting of C₁₋₆ alkyl, C₁₋₃ alkylene-S—C₁₋₄ alkyl, C₁₋₃ alkylene-aryl, C₁₋₃ alkylene-heteroaryl, C₁₋₆ alkylene-COOH, C₁₋₆ alkylene-N(R^{x})₂, and C₁₋₆ alkylene-CON (R^{x})₂, wherein each Rₓ is independently H or C₁₋₆ alkyl;

Y is an unsubstituted or substituted group selected from the group consisting of C₁₋₁₀ alkylene, C₆₋₁₀arylene, C₁₋₃ alkylene-O—C₁₋₃alkylene, O—C₁₋₆alkylene, C₁₋₃alkylene-S—C₁₋₃-alkylene, C₁₋₃alkylene-CH—CH—C₁₋₃alkylene, C₃₋₈cycloalkylene, C₁₋₃ alkylene-C₄₋₅cycloalkylene-C₁₋₃alkylene, C₁₋₃ alkylene-C₆₋₁₀arylene-C₁₋₃-alkylene, C₁₋₆alkylene-NR^{e}—, C₁₋₃ alkylene-CH(NHBoc)- and C₁₋₃ alkylene-NR^{e}—C₁₋₃ alkylene, wherein Re is selected from the group consisting of H, C₁₋₆ alkyl, C₆₋₁₀ aryl, C₁₋₃ alkylene-C₆₋₁₀aryl and CON (R^{f})₂, wherein each R^{f} is independently H or C₁₋₆ alkyl;

a and b are independently an integer ranging from 0 to 4;

c is an integer ranging from 1 to 8; d is 0 or 1; and m is an integer ranging from 1-3. In some embodiments, A is C₁₋₃alkylene, C₁₋₃alkylene-O—CO—C₂₋₅alkylene, or C₁₋₃alkylene-O—CO—C₁₋₂alkylene-O—C₁₋₂alkylene. In some embodiments, A is CH₂ or CH₂CH₂. In some embodiments, R¹ is H. In some embodiments, the amino acid moiety is derived from natural amino acid. In some embodiments, the amino acid moiety is derived from essential amino acid selected from the group consisting of phenylalanine, valine, threonine, tryptophan, methionine, leucine, isoleucine, lysine, and histidine. In some embodiments, Y is selected from the group consisting of C₁₋₅alkylene, phenylene, and C₁₋₂ alkylene-O—C₁₋₂alkylene. In some embodiments, R² and R³ in each occurrence are independently a bromine or iodine; a and b are independently 0, 1 or 2.

In some embodiments, the biocompatible polymer further includes a recurring unit of the formula II-a:

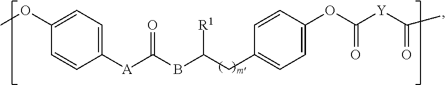

wherein m′ is an integer ranging from 1-3.

In some embodiments, the biocompatible polymer further includes a recurring unit of the formula II-b:

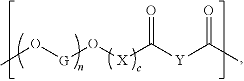

wherein G is C₂₋₃-alkylene, n is an integer ranging from 4 to 3000.

In some embodiments, the biocompatible polymer further includes a recurring unit of the formula II-c:

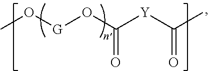

wherein G is C₂₋₃-alkylene, n′ is an integer ranging from 4 to 3000.

In some embodiments, the biocompatible polymer further includes a copolymer unit selected from the group consisting of poly(ethylene glycol), polycaprolactone-diol, polycaprolactone, poly(trimethylene carbonate), polylactide, polyglycolide, and poly(lactic-co-glycolic acid).

In some embodiments, the biocompatible polymer further includes a structure of Formula I-b,

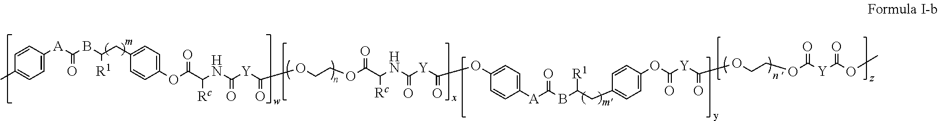

wherein w ranges from about 0.001 to 1, x ranges from 0 to 0.999, y ranges from 0 to 0.999, z ranges from 0 to 0.999, and w+x+y+z=1.000. In some embodiments, A is selected from the group consisting of C₁₋₃ alkylene, C₁₋₃ alkylene-O—CO—CH₂CH₂, C₁₋₃ alkylene-O—CO—CH₂CH₂CH₂, and C₁₋₃ alkylene-O—CO—CH₂OCH₂; B is oxygen. In some embodiments, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀. In some embodiments, R¹ and R^{c} are H.

The electrospun biodegradable polymer conduit may be constructed from PEG block polymers of any of the foregoing biocompatible polymers.

Also incorporated herein by reference in entirety are: U.S. Pat. No. 5,099,060, in particular, for its disclosure related to polycarbonate synthesis; U.S. Pat. No. 5,216,115, in particular, for its disclosure related to polyarylate synthesis; U.S. Pat. No. 6,048,521, in particular, for its disclosure related to PEG block copolymerization; and U.S. Patent Publication No. 2006/0034769, in particular, for its disclosure related to the synthesis of polymers with free acid groups.

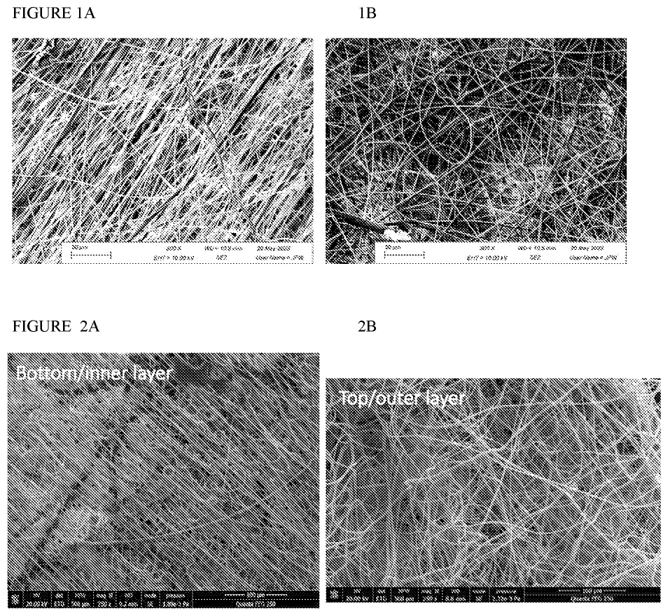

The electrospun biodegradable polymer conduit may be multilayered. It may include one layer, two layers, three layers, or four layers. Each layer may contain aligned (which may also be referred to as oriented) or unaligned (which may also be referred to as non-oriented) biopolymer fibers. The electrospun biodegradable polymer conduit may include two layers, with the inner most layer containing aligned biopolymer fibers, and the outermost layer including unaligned biopolymer fibers. FIGS. 1A, 1B, 2A and 2B are electron micrograph images of examples of bilayered sheets with oriented layers (e.g., FIGS. 1A and 2A) and unoriented layers (e.g., FIGS. 1B and 2B) of biopolymer fibers. The sheets may be rolled and fabricated as electrospun biodegradable polymer conduit of the desired diameter.

The fibroblasts (FBs) may be seeded at a defined concentration, for example, at a density of about 1.0×10⁵ cells/mL to about 5.0×10⁵ cells/mL, or about 2.5×10⁵ cells/mL, onto the exterior surface of the electrospun biodegradable polymer conduit. The fibroblasts adhere to the exterior surface and form a continuous cell layer on the exterior surface at a concentration of about 1.0×10⁵ cells/mL/cm² to about 1.0×10⁶ cells/cm². The fibroblasts may be epineurial fibroblasts. The fibroblasts may partially (about less than 50% of the surface area), substantially (greater than about 50% of the surface area), or completely (more than about 80% of the surface area) cover the exterior surface of the polymer conduit.

Fibroblasts and Schwann cells may be obtained through induction of patient cells, such as skin cells, which can be retrieved at the time of patient injury. These skin or other cells, once obtained from the patient, are differentiated into fibroblasts and Schwann cells in vitro and then applied to the electrospun biodegradable polymer conduit as described herein. Use of autologous cells reduces the likelihood of implant rejection because the conduit retains the patient's genetic material.

Using specific differentiation protocols, human Schwann cells (hSC) may be made from human induced pluripotent stem cells (iPSC) via conversion to human Schwann cell precursor cells (hSCP).

The system comprising a hydrogel matrix or augmented hydrogel matrix and Schwann cells promotes neurite regrowth from, and myelination of, injured neurons. The hydrogel matrix may be defined as a cross-linked hydrophilic polymer that does not dissolve in water and is capable of absorbing large quantities of water or other biological fluids, which may be made from RADA-16 peptides, collagen, gelatin, alginate, hyaluronic acid, or any other known hydrogel materials. The augmented hydrogel matrix is a hydrogel matrix that is combined via blending, mixing, chemical conjugation, or other known method, with another biochemical factor, such as a growth factor, guidance cue, structural peptide, adhesion factor, other chemical agent, or combination thereof.

The augmented hydrogel matrix may be a RADA16 peptide, collagen, gelatin, alginate, or hyaluronic acid hydrogel. The augmented hydrogel matrix may be functionalized with a growth factor to support cell growth, either by physical mixing with the hydrogel matrix or by chemical conjugation with the hydrogel matrix, or any combination thereof. The growth factor may be selected from Neuregulin 1 (NRG1), EGF, FGF, NGF, PDGF, VEGF, IGF, GMCSF, GCSF, TGF, Erythropoietin (EPO), TPO, BMP, HGF, GDF, Neurotrophins (e.g., GDNF, CNTF, BDNF, NT3), netrins, MSF, SGF, or any combination thereof. The growth factor may be NRG1.

The augmented hydrogel matrix may include one or more additives selected from a basal medium known for use in supporting the growth of cells (e.g., Dulbecco's Modified Eagle Medium (DMEM), fetal bovine serum (FBS), an antibiotic (e.g., penicillin, streptomycin or a combination thereof), forskolin, and any combination thereof.

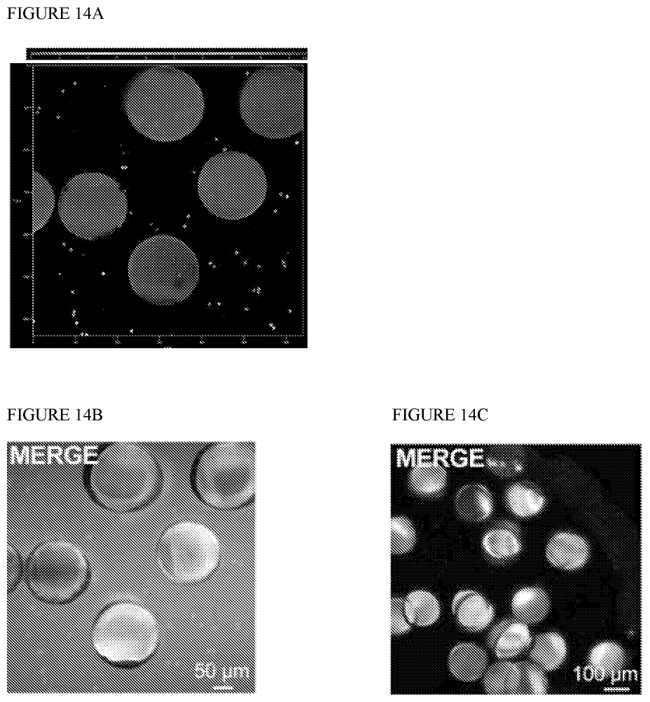

The Schwann cells may be substantially evenly distributed throughout the hydrogel matrix or augmented hydrogel matrix within the interior luminal space of the conduit. The Schwann cells may be distributed throughout the hydrogel matrix or augmented hydrogel matrix at a concentration of about 5 million cells/mL to about 80 million cells/mL, about 15 million cells/mL to about 75 million cells/mL, or about 20 million cells/mL to about 70 million cells/mL. The Schwann cells may be encapsulated by the hydrogel matrix or augmented hydrogel matrix within the interior luminal space of the conduit. FIGS. 14A-C are cross-sectional confocal microscopy images demonstrating rat Schwann cells proliferating within collagen hydrogel matrix. In these figures, tyrosine-derived polymer fibers are shown dispersed throughout the matrix, maintaining longitudinal tracts in the hydrogel matrix. In FIG. 14A, depth coding of fibers and Schwann cells are distributed in three dimensions as indicated by red-blue scale corresponding to depth within the hydrogel matrix. In FIGS. 14B and C, tyrosine-derived fibers shown as the large uniform circular structures; Schwann cell nuclei are shown as smaller points, stained with DAPI, interspersed within hydrogel matrix.

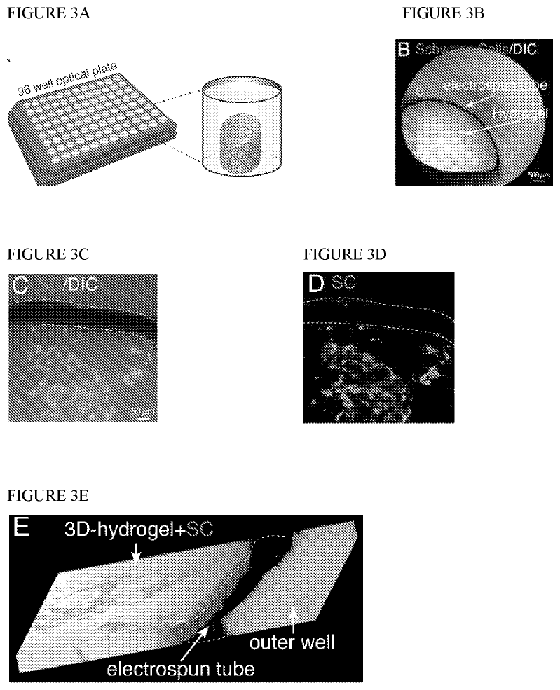

FIGS. 3A-E depict hydrogel encapsulated Schwann cells in an electrospun biodegradable polymer conduit. In FIG. 3A, a 96-well optical plate is used to hold and culture upright electrospun biodegradable polymer conduits filled with Schwann cells encapsulated in hydrogel enabling live imaging; FIG. 3B shows GFP (Green Fluorescent Protein)+ rat Schwann cells (SC) and human neural progenitor cells embedded in 3-dimensional RADA16 peptide hydrogel within the electrospun biodegradable polymer conduit. FIG. 3C is a close up panel B showing GFP+rat SC near tube edge and DIC to show conduit edge and outer well space. FIG. 3D is a close up of panel B showing only GFP+SC. FIG. 3E is an example of a 3-dimensional reconstruction of a 32 μM Z-stack showing Rat SC (GFP+) embedded in hydrogel enclosed by the electrospun biodegradable polymer conduit.

The system may also contain fibroblasts, which may be substantially evenly distributed throughout the hydrogel matrix or augmented hydrogel matrix within the interior luminal space of the conduit. The fibroblasts may be present at a lower concentration than the Schwann cells, for example at about a 2:10 to about 1:20, or about 1:10 ratio of fibroblasts to Schwann cells. The fibroblasts facilitate Schwann cell function within the cellularized nerve regeneration graft, particularly at the level of the endoneurium, the innermost connective tissue layer found within nerve fascicles that surround myelinated axons. The system may contain other supporting cells, optionally in addition to fibroblasts, such as, but not limited to, endothelial cells, or other cells to support the graft cellular architecture. In an embodiment, the inner luminal space of the polymer conduit may be filled with hydrogel matrix or augmented hydrogel matrix with rat SC growing throughout. When present, GFP+ fibroblasts may be only seeded on the exterior surface of the polymer conduit, therefore no GFP signal should be present within the inner luminal space of the polymer conduit.

Within the hydrogel matrix or augmented hydrogel matrix, fibers or hollow tubes, optionally having a diameter of about 5 μm to about 50 μm, or about 20 μm, may be added to reproduce an endoneurial sheath-like substructure. The fibers or hollow tubes may be made from collagen, or other suitable fibers, including fast-degrading polymeric fibers, or water-soluble sacrificial fibers made of materials, such as sucrose or other suitable saccharide, or made by creating channels with metallic or polymeric wires. Creation of an endoneurial sheath-like substructure may augment cell-cell signaling during graft development and after implantation.

Another embodiment is a method of making a cellularized nerve regeneration graft. The nerve call graft comprising: an electrospun biodegradable polymer conduit having an exterior surface and an interior luminal space; a plurality of fibroblasts seeded to the exterior surface of the conduit; and a system filling the interior luminal space of the conduit, the system comprising a hydrogel matrix or augmented hydrogel matrix and Schwann cells. The method may include: electrospinning a polymer formulation, e.g., a tyrosine-derived or tyrosol-derived polymer, to make the electrospun biodegradable polymer conduit; culturing fibroblasts; seeding the fibroblasts on the exterior surface of the electrospun biodegradable polymer conduit; generating human Schwann cells; embedding the human Schwann cells in the hydrogel matrix or augmented hydrogel matrix to make the system; seeding the system in the interior luminal space of the electrospun biodegradable polymer conduit to make a seeded conduit; and incubating the seeded conduit for about 1 to about 6 weeks, or about 2 to about 4 weeks in the system to make the cellularized nerve regeneration graft.

The fibroblasts may be cultured by any method known in the art. The fibroblasts may be epineurial fibroblasts. The exterior surface of an electrospun tyrosine-derived polymer conduit may be seeded with epineurial fibroblasts. The exterior surface of an electrospun tyrosol-derived polymer conduit may be seeded with epineurial fibroblasts

Generating human Schwann cells may be accomplished by any means known in the art. After fibroblasts are seeded on the exterior surface, the generated human Schwann cells may be embedded in the hydrogel matrix or augmented hydrogel matrix, which is optionally functionalized with one or more growth factors to support axonal regrowth, Schwann cell proliferation, and myelination of axonal projections. A hydrogel matrix or augmented hydrogel matrix at a defined concentration (e.g., about 0.1% to about 0.4%, or about 0.25%) may be mixed with Schwann cells (SCs) at a defined concentration (e.g., about 1 million cells/mL to about 20 million cells/mL, about 2 million cells/mL to about 15 million cells/mL, about 5 million cells/mL to about 10 million cells/mL or about 10 million cells/mL) to create a system. The system may be injected into the interior luminal space of the electrospun biodegradable polymer conduit, thus filling the entire interior luminal space. The filled electrospun biodegradable polymer conduit may be incubated in the system for about 2 to about 4 weeks, or about 3 weeks, to allow both epineurial fibroblasts and Schwann cells to proliferate within the conduit. A media solution may be added and routinely replaced during the incubation. The media solution may be Dulbecco's Modified Eagle Medium (DMEM), fetal bovine serum (FBS), an antibiotic (e.g., penicillin, streptomycin or a combination thereof), and any combination thereof. The media solution may be DMEM/10% FBS (Fetal Bovine Serum)/1% Penicllin/Streptomycin, optionally supplemented with forskolin and/or a growth factor at defined concentrations. Forskolin may be added at a concentration of about 1 μM to about 5 μM, or about 2 μM. The growth factor may be neuregulin-1, and may be added at a concentration of about 5 ng/ml to about 15 ng/ml, or about 10 ng/ml. The media solution may be 1.2 mL DMEM/10% FBS/Penicillin/streptomycin.

During incubation, the fibroblasts and Schwann cells may be grown to confluence in the media solution such that, at the end of this period, the fibroblasts constitute an outer contiguous cell layer at a defined concentration of about 1.0×10⁵ cells/mL/cm² to about 1.0×10⁶ cells/cm² on the exterior surface of the conduit while, in the interior luminal space, there may be a confluent, 3D, substantially even distribution of Schwann cells throughout the hydrogel matrix or augmented hydrogel matrix such that the Schwann cells occupy the entire luminal space of the conduit at a defined concentration of about 5 million cells/mL to about 80 million cells/mL, about 15 million cells/mL to about 75 million cells/mL, or about 20 million cells/mL to about 70 million cells/mL.

To seed the fibroblasts on the exterior surface of the electrospun biodegradable polymer conduit, a microdevice may be made by 3-D printing and assembled. The 3-D printed microdevice may include a number of parts including one or more rods and one or more gears, which are constructed as separate pieces but which may be assembled, or reversibly or irreversibly interlocked, to create a microdevice for seeding the fibroblasts.

A 3-D printed microdevice may also be used to seed the system in the interior luminal space of the electrospun biodegradable polymer conduit. The 3-D printed microdevice may be the same microdevice used for seeding the fibroblasts.

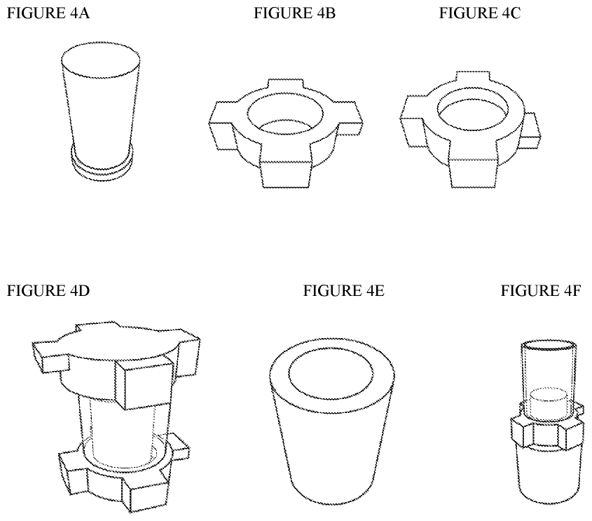

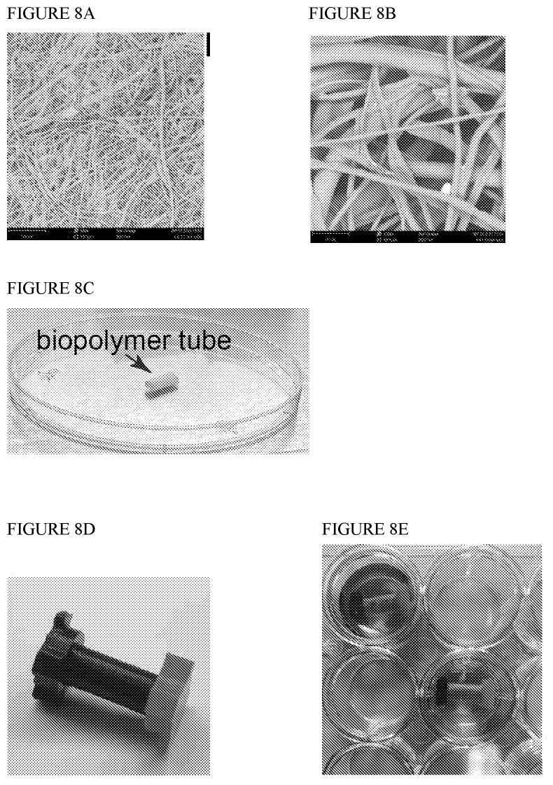

For example, as shown in FIG. 4, a microdevice may be assembled from a rod (FIG. 4A), a hollow gear (FIG. 4B), a solid gear (FIG. 4C), and capped hollow tube (FIG. 4E). To assemble the microdevice, the rod may be inserted through hollow gear. The rod may have a diameter of about 1.0 mm to about 4.0 mm, depending on the application, with an overall length of about 5.0 mm to about 5.0 cm, or greater. Each of the hollow gear and solid gear may have a diameter of about 8.0 mm to about 2.0 cm with a thickness of about 1.0 mm to about 2.0 mm. Each of the components of the 3-D printed microdevice may be constructed from polylactic acid (PLA), polycaprolactone (PCL), or any other material known for use in the art. The electrospun biodegradable polymer conduit may be then placed over (or around) the rod and capped with the solid gear, resulting in an assembly, as shown in FIG. 4D, with the electrospun biodegradable polymer conduit shown in transparent shading. The assembly, as shown in FIG. 3D, may then placed into a well plate (shown below in FIG. 8E) and seeded with fibroblast suspension at a determined concentration, e.g., about 1.0×10⁵ cells/mL to about 5.0×10⁵ cells/mL, or about 2.5×10⁵ cells/mL. After being submerged in the culture media within the well plate, the assembly may be permitted to rest for about 2 to about 4 minutes to allow the fibroblasts to attach to the scaffold and then the assembly may be rotated about its longitudinal axis by 90 degrees while still inside of the well. This process may be repeated three times until four arc lengths of the conduit is seeded with fibroblast suspension. Optionally, the assembly may be then submerged in a media solution by pipetting fresh media into the bottom of the well until the assembly was completely covered by media.

After seeding the exterior surface with fibroblasts, which may take a period of about 1 hour to about 24 hours, the assembly may be removed from the well and placed upright, with the hollow gear resting on a capped hollow tube in an adjacent well. The solid gear may be removed from the assembly and, optionally, discarded or reused after sterilization. Pressure may be applied from above to the rod which forces the rod through the hollow gear and into the capped hollow tube, resulting in the interior of the biopolymer tube (or conduit) being exposed, as shown in FIG. 4F. The device shown in FIG. 4F may then be placed into any known culture tube, such as a polystyrene culture tube, and the system (e.g., the mixture of the hydrogel matrix or augmented hydrogel matrix and Schwann cells) may be inserted into the interior luminal space of the electrospun biodegradable polymer conduit that has been seeded with fibroblasts on the exterior surface. Optionally, a media solution may be added to the culture tube to submerge the device. The culture tube may be capped and incubated, for example at about 33-40 degrees C. and about 3%-7% CO₂, or about 37 degrees C. and about 5% CO2 for about 15 to about 30 minutes, or about 20 minutes. After a first period of incubation, at least a portion of the media solution may be replaced, and this process may be repeated 2-4 times every about 15 to about 30 minutes, or replaced about every 20 minutes. Incubation may then be permitted for about 3 weeks, with at least a portion of the media solution in the culture tube being replaced about every 2-3 days.

Once the fibroblasts and Schwann cells have grown to confluence, the filled and seeded electrospun biopolymer conduit is a solid graft-like structure containing Schwann cells throughout the lumen, with fibroblasts localized externally. Structurally, the fibroblasts may form the outer epineurial sheath, a layer of connective tissue that encloses fascicles of peripheral nerves. The fibroblasts serve the purpose of directing the apical/basal orientation of Schwann cells while also secreting growth factors to support Schwann cell survival in vitro and in vivo.

The terms used in connection with this embodiment (i.e., method of making) have the same meanings and definitions as discussed above.

A method is disclosed for repairing an injured peripheral nerve comprising implanting a cellularized nerve regeneration graft into a patient by adhering the cellularized nerve regeneration graft to a distal end and a proximal end of the injured nerve injury. The cellularized nerve regeneration graft comprises: an electrospun biodegradable polymer conduit having an exterior surface and an interior luminal space; a plurality of fibroblasts seeded to the exterior surface of the conduit; and a system filling the interior luminal space of the conduit, the system comprising a hydrogel matrix or augmented hydrogel matrix and Schwann cells. The Schwann cells, which occupy the interior of the cellularized nerve regeneration graft, serve the purpose of supporting in-growing axons by secreting signaling molecules and growth factors, including ciliary neurotrophic factor (CNTF), to guide axon growth through the graft and to myelinate regenerating axons once the graft is implanted into the patient.

A method of forming a plurality of channels within a hydrogel matrix or augmented hydrogel matrix is disclosed. In this method, Schwann cells are cultured onto resorbable, also referred to as dissolvable, fibers (which may be about 10 μm to about 50 μm in diameter) to create a plurality of channels within a hydrogel matrix or augmented hydrogel matrix, optionally within a biodegradable polymer conduit. The resorbable fibers may be made of: poly(lactic-co-glycolic acid) (PLGA), polyglycolic acid (PGA), a tyrosine-derived or tyrosol-derived polymer with a high concentration of poly(ethylene glycol) (PEG), or any combination thereof. One or more coating materials may be applied to the surface of the resorbable fibers. The coating material may be selected from: poly-d-lysine or other charged molecules; macromolecular coatings such as collagen hydrogel, hyaluronic acid or others which may mimic the constituents of the extracellular matrix; and any combination thereof. Cells in suspension may be applied to the resorbable fibers and may be assisted by the action of capillary action to coat the fibers evenly. FIG. 13 is an example of 10× magnification imaging of rat Schwann cells growing on E1001k fibers of approximately 50 μm.

The resorbable fibers cultured with the Schwann cells may be loaded into a biodegradable polymer conduit in a longitudinal arrangement (alternatively, the fibers may be present within the biodegradable polymer conduit at the time of cell seeding), with a spacing between the fibers of about 20 μm to about 100 μm. The remaining luminal space may be filled with the hydrogel matrix or augmented hydrogel matrix, such that the interior consists of parallel-oriented fibers surrounded by Schwann cells, or with Schwann cells directly adhered to the resorbable fibers, and the remainder of the luminal space occupied by the hydrogel matrix or augmented hydrogel matrix, or a hydrogel matrix or augmented hydrogel matrix mixed with cells, such as but not limited to, Schwann cells, fibroblasts and/or other cells. When mixed with cells (for example, this may be a cell suspension with culture media having density of approximately 5 million cells/mL to 80 million cells/mL), the remaining luminal space may be filled with the hydrogel matrix or augmented hydrogel matrix and cells mixture, optionally having a ratio of approximately 3:2 (hydrogel:cells) in volume.

After the passage of time, e.g., about 1 week to about 6 months, the resorbable fibers dissolve, leaving Schwann cells within channels running longitudinally within the hydrogel matrix or augmented hydrogel matrix. The resorbable fibers may also constitute such a composition that an inner, quickly dissolving material, such as, but not limited to, sucrose, poly(vinyl alcohol) (PVA), poly(ethylene glycol) (PEG), or a combination thereof, is coated with resorbable material, such as, but not limited to, PLGA, PGA, tyrosine-, or tyrosol-derived polymers with large amounts of PEG, or a combination thereof which dissolves over a longer time period such that the arrangement results in a hollow channel with adherent/surrounding cells. These channels serve as the template endoneurium for in-growing axons during nerve regeneration, with Schwann cells lining the tracts to myelinate in-growing axons. The hollow channels may contain Schwann cells within the longitudinal space. This method may be employed to create channels without Schwann cells, such that nutrients and culture media can traverse the length of the conduit and diffuse throughout the hydrogel matrix or augmented hydrogel matrix.

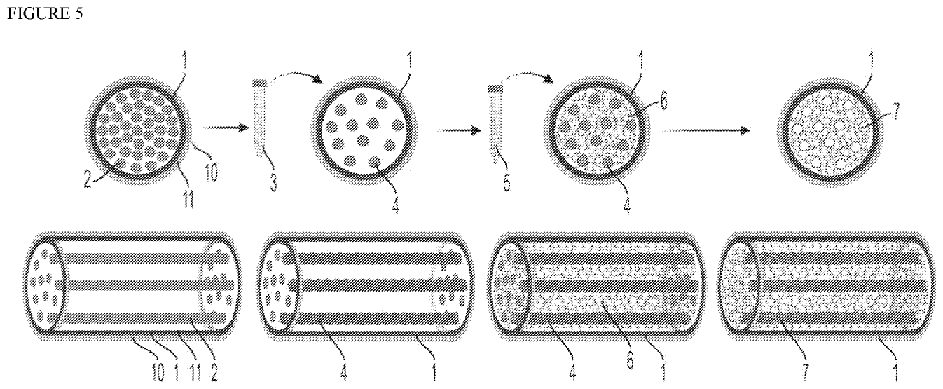

FIG. 5 is an example of the method of forming a plurality of channels reinforced with Schwann cells within a hydrogel matrix or augmented hydrogel matrix. As shown therein, a biodegradable polymer conduit 1, also referred to as a scaffold tube, is shown filled with resorbable fibers 2. Next, a suspension 3 with Schwann cells is seeded onto the resorbable fibers and the cells allowed to adhere to the fibers to make SC coated fibers 4. After about 4 hours to about 5 days, the remaining luminal space within the biodegradable polymer conduit is filled with a suspension of hydrogel or augmented hydrogel matrix, optionally including Schwann cells 5. Once added, this leaves the biodegradable polymer conduit 1 filled with hydrogel encapsulated Schwann cells 6 surrounding the Schwann cells coated fibers 4. After about 3 weeks, the resorbable fibers degrade leaving channels 7 reinforced by Schwann cells, with the channels running longitudinally and for in-growing axons during nerve regeneration. As shown in FIG. 5, the biodegradable polymer conduit 1 includes an outer unoriented electrospun layer 10 and an inner oriented electrospun layer 11.

The features and advantages of the present disclosure are more fully shown by the following examples which are provided for purposes of illustration, and are not to be construed as limiting the invention in any way.

### EXAMPLES

### Example 1

Preparation of Tyrosine-derived polymer electrospun scaffold: Scaffolds were prepared from the random block copolymer poly(DTE-co-10% DT-co-1% PEG carbonate) composed of desaminotyrosyl-tyrosine ethyl ester (DTE), desaminotyrosyl-tyrosine (DT), and polyethylene glycol (PEG), that will be referred to as E1001 (1k), where 10 and 01 are the mole percent of DT and PEG, respectively, and 1k is the molecular weight of PEG (1000 Da)^{5,6}. The three components of the polymer serve different purposes. The main chain of DTE segments aids in polymer processing, has the required mechanical properties, and provides chemical stability during processing and use. Increasing the fraction of DT units increases the degradation rate from days at 25 mol % DT to hours at 50 mol % DT. 10 mol % DT used here provides a degradation rate of approximately 1 year. Because both DTE and DT are very hydrophobic, PEG was incorporated to increase water content and allow for degradation. PEG (1k) remains biocompatible after degradation and, unlike PEG (2k), does not crystallize in the scaffold. The polymer was dissolved in hexafluoropropylene to prepare a 16% solution. The electrospinning apparatus consisted of a syringe pump (kd Scientific, Model 780100, Holliston, MA), high voltage DC power supply (Gamma High Voltage Research, Model ES30P/5 W/DAM, Ormand Beach, FL) fitted with an 18 G needle, and a rotating mandrel. The syringe was placed 10 cm away from the mandrel.

Rat Schwann cell culture: Primary rat Schwann cells (SC) were cultured on Matrigel (Corning) coated plates in DMEM (Dulbecco's Modified Eagle's Medium) supplemented with 10% FBS, 1% Penicillin/Streptomycin, 2 μM Forskolin, and 10 ng/ml Neuregulin-1 (NRG1). Rat SC were routinely passaged using Accutase (StemCell Technologies).

Generation of induced Schwann cells from human induced pluripotent stem cells (iPSC) and/or from human fibroblasts: To generate human induced Schwann cells, human iPSC were passaged with Accutase (StemCell Technologies) and plated on growth-factor-reduced Matrigel (Corning) plates in induction medium containing 1:1 DMEM/F12 (Hyclone) and neurobasal medium (Gibco) supplemented with 1× B27 (Gibco), 3 μM CHIR99021 (StemCell Technologies), 20 μM SB431542 (StemCell Technologies), and 50 ng/ml Neuregulin-1 (Peprotech) for 18 days with media changes every other day. At day 18 the media was changed to 1:1 DMEM/F12 and neurobasal medium supplemented with 1× B27, 200 ng/ml Neuregulin-1, 4 μM Forskolin (Sigma), 10 ng/ml PDFG-BB (Peprotech), and 100 nm all-trans retinoic acid (Sigma) for 3 days. After 3 days, the same medium was given minus the all-trans retinoic acid and forskolin and cultured for 3 more days. The induced Schwann cells were then maintained in 1:1 DMEM/F12 and neurobasal medium supplemented with 1× B27 and 200 ng/ml Neuregulin-1 until ready for experiments and fed every 3-4 days. By ectopic expression of pro-Schwann cell developmental transcription factors such as SOX10, KROX20, or other transcription factors human fibroblasts may be directly inducted into human Schwann cells.

Encapsulation of Schwann cells in hydrogel: To encapsulate Schwann cells in RADA16 peptide hydrogel, either rat Schwann cells or human-induced Schwann cells were dissociated with Accutase, resuspended in 10% sucrose water, and counted with a hemocytometer to ensure proper loading density of about 1 million cells/ml to about 20 million cells/ml. RADA16 peptide hydrogel was mixed 1:1 with 20% sucrose water. The cell suspension and hydrogel mixture were then combined 1:1, briefly mixed, and loaded in an upright electrospun biodegradable polymer conduit. The final concentration of the hydrogel is 0.25%. Immediately after loading with the hydrogel cell mixture, media composing DMEM, 10% FBS, 1% Penicillin/Streptomycin, 2 μM Forskolin, and 10 ng/ml Neuregulin-1 was added to the well to initiate curing of the hydrogel. Media was replaced after the first 20 minutes to reduce acute acidity caused by the hydrogel. Electrospun biodegradable polymer conduits containing the hydrogel encapsulated Schwann cells were then cultured in an incubator at 37° C. 5% CO₂. Media was replaced every 1-2 days, which may be accomplished by means of a continuous flow reactor loop.

### Results

### Overall Design of Cellularized Nerve Regeneration Graft (CNRG)

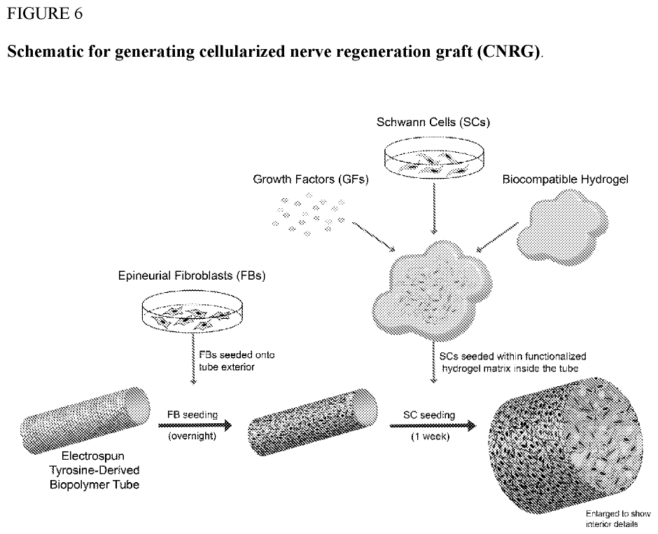

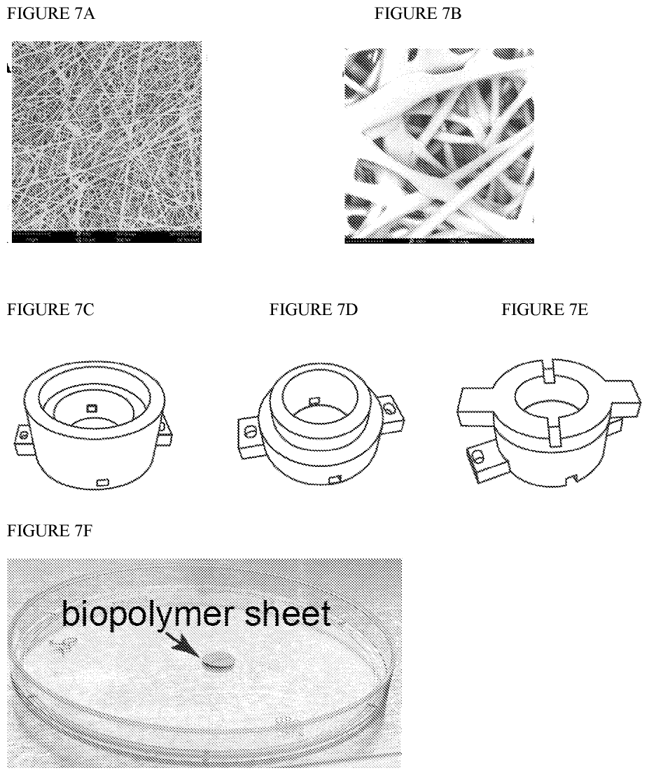

The goal is to construct a cellularized nerve regeneration graft that will be used for reconstructing and repairing peripheral nerve damage. The overall design of the graft is depicted in FIG. 6. Briefly, 1) construct a biopolymer conduit (tube) using electrospun tyrosine-derived polymer as the substrate; 2) culture epineurial fibroblasts (FBs) and seed them on the outer surface of the conduit using the microdevice that was developed (see FIGS. 7C-E and 4A-F); 3) generate Schwann Cells (SCs), which are the myelinating cells for peripheral nerves, and coat them onto resorbable fibers and/or mix them with functionalized hydrogel (with growth factors); and 4) seed the mixture of hydrogel/SCs in the inner space of the biodegradable polymer conduits using the microdevice shown in FIG. 7C-7E.

The cellularized nerve regeneration graft will be cultured in culture dishes for about 1-6 weeks before grafting into the lesion site to help facilitate axonal regeneration, myelination, and function.

### Manufacturing of Biopolymer and Assemblies for Cellularized Nerve Regeneration Graft

Flat sheets were made using a large diameter (5 cm) mandrel that was laterally oscillated to obtain 13×21 cm mats. The speed of the mandrel was controlled by a DC power supply (Model 1627A, BK Precision, Yorba Linda, CA). The linear speed was set at 30 meters per minute (mpm) for unaligned layers and 650 mpm for aligned layers.

Multi-layered scaffolds were prepared in three steps: 16% polymer solution was spun into an unaligned layer at 2 mL/h for 30 minutes, followed by an additional unaligned layer with 10% solution at 1 mL/h for 30 minutes, and finally an aligned layer with 10% solution at 1 mL/h for 30 minutes.

Initially, these multilayer scaffolds were electrospun as flat sheets, dried slowly at RT (Room Temperature), and refrigerated at 4 degrees C. until needed. Shortly prior to culture experiments, the polymer was cut into 8-mm circular sections (as shown in FIG. 7F), as needed, using an 8-mm diameter steel hollow punch. Cut scaffolds were secured into the snap-fit fixture shown in FIG. 7C-7E after sterilizing all components under UV light for 30 minutes.

Following proof of cell compatibility with the flat biopolymer sheets, hollow biopolymer conduits were then prepared using smaller diameter (1.5, 2, and 4 mm) mandrels. The mandrels were coated with PEG gel to facilitate the release of the conduit after electrospinning. These mandrels were mounted onto a chuck (IKA, model R20DS1) and spun at approximately 200 rpm for 10 min to 2 h to obtain tubes of different wall thicknesses and tube diameters. After electrospinning, the mandrel was removed from the chuck, wetted slightly with deionized water, and the polymer conduit was carefully removed by sliding it off the mandrel. Conduits were allowed to dry slowly and then refrigerated at 4 degrees C., until needed, to prevent decomposition. For culture experiments, shortly prior to cell seeding the conduits were cut into 5-mm length sections using stainless steel surgical scissors and then sterilized under UV light for 30 minutes. Scaffold thickness was measured using a micrometer. Fiber morphology was assessed using a scanning electron microscope (SEM) (Phenom ProX, Nanoscience Instruments, Phoenix, AZ).

SEM image of unaligned E1001 (k) biopolymer fibers forming flat electrospun sheet, at 500× magnification is shown in FIG. 7A. SEM image of the same electrospun sheet in FIG. 7A but at 5000× magnification is shown in FIG. 7B. FIG. 7C shows a bottom half of 3D-printed snap fixture for holding flat biopolymer sheet. An 8-mm diameter piece of electrospun E1001 (k) scaffold may be placed into the round inset shown within the device. FIG. 7D shows a top half of 3D-printed snap fixture. Once the electrospun biodegradable polymer sheet is placed into the bottom half piece, the top half piece is snapped in, thus securing an electrospun biopolymer sheet in place. This fully assembled configuration is shown in FIG. 7E. Once the device is fully assembled, cell suspension can be pipetted into the wells created on either side of the snap fixture. This allows cells to adhere to only one side of the scaffold fibers while preventing cell migration to the other side. The device is sized with an outer width (from tab to tab) of about 12 mm such that the entire device fits into, and can be turned inside, a well of a standard 24-well culture dish. Holes through the side walls allow for culture media to flow through the device. Indentations in the tabs allow for handling of the device using forceps. FIG. 7F is a sample circular 8-mm DTE biopolymer sheet.

To seed cells onto the exterior and interior of the electrospun biodegradable polymer conduit, also referred to herein as an electrospun biopolymer tube or an electrospun biodegradable polymer conduit, a device was assembled such that: a rod (FIG. 4A) was inserted through hollow gear (FIG. 4B). The electrospun biopolymer tube was then placed over the rod and capped with solid gear (FIG. 4C), resulting in an assembly, as shown in FIG. 4D, with the electrospun biopolymer tube here shown in transparent shading. The assembly was then placed longitudinally into a 24-well plate (shown below in FIG. 8E) and seeded with 40 μL of fibroblast suspension at a density of 2.5×10⁵ cells/mL by pipetting the volume across the length of the exterior of the exposed electrospun biopolymer tube. The cells were allowed to attach to the scaffold for 2 minutes and then the device was rotated about its longitudinal axis by 90 degrees by applying force to the tab of one of the gears with a pair of forceps while the device was still inside of the well. This process was repeated three times until four arc lengths of the tube were seeded with fibroblast suspension. The assembly was then submerged in 1.2 mL DMEM/10% FBS/1% P/S by pipetting fresh media into the bottom of the well until the assembly was completely covered by media. After fibroblast cell attachment overnight, the assembly was removed intact from the well with a pair of forceps and placed upright, with the hollow gear (shown in FIG. 4B) resting on a capped hollow tube (shown in FIG. 4E) in an adjacent well of the 24-well plate. The solid gear was then removed from the assembly with a pair of forceps and discarded. Using the forceps, pressure was applied directly from above to the rod which forced the rod through hollow gear and into the capped hollow tube, resulting in the interior of the electrospun biopolymer tube being exposed, as shown in FIG. 4F. The entire device was then placed carefully into a polystyrene culture tube and a hydrogel-Schwann cell mixture was pipetted into the interior lumen of the electrospun biopolymer tube. Immediately after dispensing the cell mixture, 1.5 mL of culture media was added to the polystyrene tube to submerge the entire device. The tube was then capped and incubated at 37 degrees C. and 5% CO2 for 20 minutes. After 20 minutes 1.0 mL of the media was replaced, and this process was repeated after 40 minutes. The culture media in the tube was replaced similarly every 2-3 days over the course of three weeks.

### Construction of Tyrosine-Derived Biopolymer Tube and Fixtures for Seeding Cells in Inner Compartment and Outer Layer

Using the method described above, a tyrosine-derived biopolymer tube was constructed, as shown in FIG. 8. FIG. 8A is an SEM image of exterior surface of biopolymer conduit, 500× magnification. FIG. 8B is an SEM image of the exterior surface of the same biopolymer conduit as in FIG. 8A, but at 5000×. FIG. 8C is a 10-mm length of tyrosine-derived polymer conduit. FIG. 8D is a photograph of a gear and rod assembly as shown in FIG. 4D (here shown without biopolymer conduit). FIG. 8E is a photograph of a gear and rod assembly with biopolymer conduit seeded with fibroblasts submerged in culture media within a standard 24-well plate. A volume of 40 μL of fibroblast suspension was pipetted across the length of the exposed polymer conduit at a density of 2.5×10⁵ cells/mL.

### Generation of Human Schwann Cells

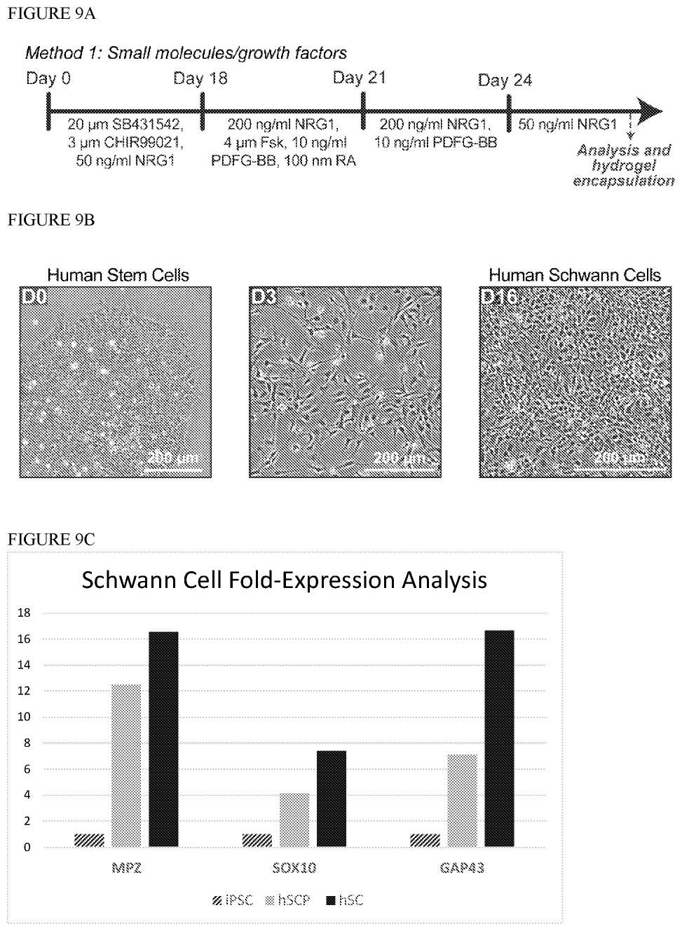

To generate human Schwann cells for incorporation in the cellularized nerve regeneration graft, Schwann cells were differentiated from human induced pluripotent stem cells (iPSCs) using a previously published method (see Kim, H S., et al., “Directly induced human Schwann cell precursors as a valuable source of Schwann cells,” Stem Cell Res Ther 11, 257 (2020); https://doi.org/10.1186/s13287-020-01772-x) shown in FIG. 9A: this method uses small molecule and growth factors to differentiate Schwann cells from patient iPSCs. Briefly, iPSCs are plated on growth-factor-reduced Matrigel plates in a cocktail of 20 μM SB431542, and 50 ng/ml Neuregulin-1 for 18 days. At day 18 the cocktail was changed to 200 ng/ml Neuregulin-1, 4 μM Forskolin, 10 ng/ml PDFG-BB, and 100 nm all-trans retinoic acid for 3 days. After 3 days, the same medium was given minus the all-trans retinoic acid and forskolin and cultured for 3 more days. The induced Schwann cells were then maintained in 50 ng/ml Neuregulin-1 until ready for experiments. FIG. 9B shows brightfield images of cells at various stages of the differentiation process. These are representative cell culture images of Schwann cell induction at Day 0 (human stem cells), Day 3 (Schwann cell precursors (hSCP)), and Day 16 (hSC) using this method.

Cell-specific biomolecular markers are used to identify the conversion of iPSCs to human Schwann cells. Myelin protein zero (MPZ) (myelin protein expressed by Schwann Cells), SOX10 (transcription factor important for neural crest cell progression and Schwann Cell differentiation) and GAP43 (expressed by Schwann cell precursors and non-myelinating Schwann Cells) for each of the cells at Day 0, 3 and 16 were analyzed and graphed as shown in FIG. 9C. As evident from the data, expression of the cells as an indicator of the presence of Schwann cells increased over time.

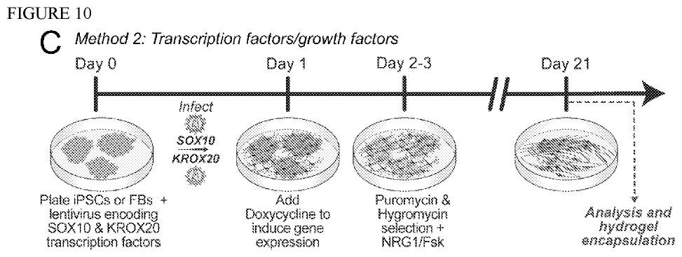

A second published method of Schwann cell induction using transcription factors to drive the induction of human Schwann cells (see Mazzara, P., et al., “Two factor-based reprogramming of rodent and human fibroblasts into Schwann cells,” Nat Commun 8, 14088 (2017); https://doi.org/10.1038/ncomms14088) was also tested; Method 2 uses transcription factors delivered by lentivirus in combination with growth factors to drive differentiation of either iPSCs or fibroblasts (FBs) to Schwann cells. As depicted in FIG. 10, SOX10 and KROX20 transcription factors are delivered to either human iPSCs or fibroblasts and then induced by the addition of doxycycline for two weeks. Additionally, Neuregulin-1 and Forskolin are added during this process to encourage the differentiation of human Schwann cells.

### Survivability and Insulation Tests of Electrospun Biopolymers

A condition was to demonstrate that an electrospun biodegradable polymer scaffold, derived from tyrosine esters, can support the growth of human fibroblasts and rat Schwann cells in vitro. To prove cell compatibility with the polymer scaffold, human fibroblasts were grown onto one side of a multilayered E1001 (k) polymer sheet. The fibroblasts were seeded at an initial concentration of 2.5×10⁵ cells/mL, such that 100 μL of cell suspension fibroblasts in DMEM/10% FBS/1% P/S was pipetted into the wells of the device shown in FIG. 8E for an initial seeding area density of 1.0×10⁵ cells/mL/cm².

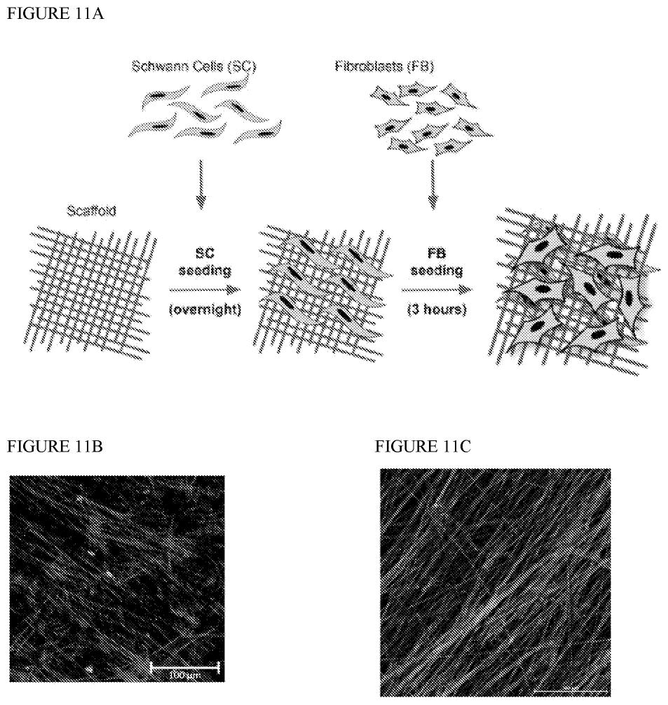

FIGS. 11A-C show a diagrammatic representation of Schwann cells and fibroblasts cultured onto opposite sides of an electrospun biodegradable polymer scaffold. In FIG. 11A, one cell type (here Schwann cells) is first cultured onto the top layer of the fibers. Once these cells adhere to the fibers, the scaffold is then inverted, and another cell type (here fibroblasts) is cultured onto the bottom layer of the scaffold. The intervening scaffold localizes the cells to their respective sides while preventing migration of either cell to the opposing side. In FIG. 11B, human fibroblasts have grown onto the unaligned fibers of the biopolymer scaffold; note the multidirectional projects of the actin cytoskeleton as shown by Texas Red phalloidin stain. In FIG. 11C, rat Schwann cells (blue nuclei and red actin cytoskeleton) are shown growing in a linear alignment along the biopolymer fibers shown in green. Such alignment of varying cell types demonstrates the ability to localize and control cell growth and proliferation using the biopolymer scaffold presented herein.

### Constructing the Cellularized Nerve Regeneration Graft

Having demonstrated that both SCs and FBs can grow on the biodegradable polymer, our next goal is to construct a transplantable cellularized nerve regeneration graft in a 3D format. As described in the overall scheme (FIG. 6), epineural FBs are cultured on the outer surface of the biopolymer tube and SCs are cultured in functionalized hydrogel and the hydrogel seeded inside the tube to form a solid structure with SCs cultured in the 3D matrix inside the tube, the biopolymer walls and then the epineural FBs on the outside wall of the biopolymer.

Grafting the Cellularized Nerve Regeneration Graft into Peripheral Nerve Injury Animal Model

For proof-of-principle use of the CNRG, a further test will be to confirm that CNRG can help repair peripheral nerve injury in animal models. Specifically, B6/C₅₇ mice will be deeply anesthetized with isoflurane; the left-side sciatic nerve will be exposed, and a 5 mm lesion gap will be made in nerve. The removed nerve gap will be replaced with a similar sized CNRG, and the biopolymer layer will be sutured together with the epineurium connective tissue membrane of the sciatic nerve. The distal end of the CNRG hydrogel has nerve growth factors (10 ng/ml Brain-Derived Neurotrophic Factor (BDNF), 10 ng/ml Neurotrophin-3 (NT3), and 10 ng/ml Glial-Derived Neurotrophic Factor (GDNF)). Post-surgical care will be taken in these animals such as antibiotics (amoxicillin) and analgesic (buprenorphine). The animal condition will be monitored every 6 hours and the animal walking gait will be monitored; sensory sensation in the left hind paw will be monitored using Von Frey filaments. The recovery of the injured animal will be compared with a) sham surgery animals (mice with skin incision but not nerve cut), and b) animals with 5-mm lesion gap repaired with autograft.

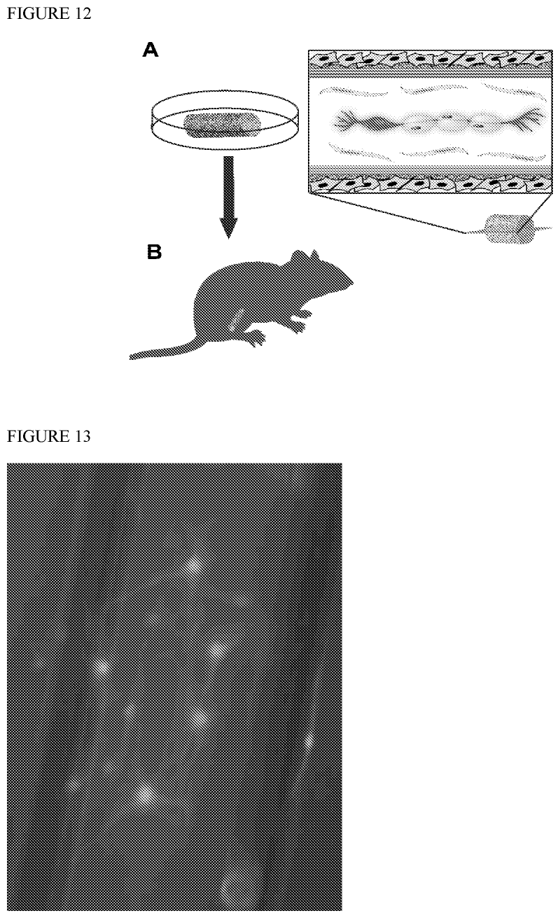

FIG. 12 depicts a general scheme for implantation of CNRG into a murine peripheral nerve injury model. It shows in vitro modeling of axon growth through the CNRG. Axonal projections are expected to grow through the hydrogel and become myelinated by Schwann cells. Fibroblasts and non-myelinating Schwann cells will secrete ECM (extracellular matrix) components and growth factors to distinct layers of the CNRG. Once cells have grown to confluence within the CNRG, it will be surgically inserted into a murine peripheral nerve injury model. Recovery of motor and sensory capabilities will be assessed in CNRG-treated, autograft-treated and sham animals.

In conclusion, the goal of the study was to construct the CNRG that is suitable for reconstructing and repairing peripheral nerve damage. The following objectives were accomplished through this initial study: 1) To prove fibroblast and Schwann cell compatibility with an electrospun tyrosine-derived biopolymer E1001 (k), cells were successfully cultured onto flat sheets of the electrospun biopolymer; 2) To prove distinct localization and organization of cells, fibroblasts and Schwann cells were cultured on opposite sides of a flat biopolymer sheet such that they adopted distinct layers without crossing the biopolymer fiber layer and adopted a specific alignment based on the alignment of the polymer fibers; 3) To prove Schwann cell-hydrogel compatibility, Schwann cells were successfully cultured in a hydrogel matrix and live-imaged to demonstrate their survival and proliferation in a three-dimensional hydrogel matrix; 4) To prove the ability to grow cells within the lumen of a biopolymer tube, Schwann cells were mixed with a hydrogel matrix or augmented hydrogel matrix and injected into the interior of a 3.5 mm diameter electrospun tube with a length of 5.0 mm-survival and proliferation of Schwann cells was confirmed via confocal fluorescent and differential interference contrast (DIC) microscopy; 5) To prove the ability to culture Schwann cells onto biodegradable fibers; and 6) To prove the final architecture of the CNRG, a biopolymer tube was first seeded with fibroblasts on its exterior surface and then filled with hydrogel encapsulated Schwann cells in the manner described in point “4)”, after which survival of both fibroblasts and Schwann cells on the exterior surface and throughout the interior lumen of the tube was confirmed via imaging and cryo-sectioning.

While there have been described what are presently believed to be various aspects and certain desirable embodiments of the disclosure, those skilled in the art will recognize that changes and modifications may be made thereto without departing from the spirit of the disclosure, and it is intended to include all such changes and modifications as fall within the true scope of the disclosure.

Although the invention has been described in detail with reference to certain preferred embodiments, variations and modifications exist within the scope and spirit of the invention as described and defined in the following claims.

1. A cellularized nerve regeneration graft comprising:
a. an electrospun biodegradable polymer conduit having an exterior surface and an interior luminal space;
b. a plurality of fibroblasts seeded to the exterior surface of the conduit; and
c. a system filling the interior luminal space of the conduit, the system comprising a hydrogel matrix or augmented hydrogel matrix and Schwann cells.

2. The cellularized nerve regeneration graft of claim 1, wherein the electrospun biodegradable polymer conduit comprises a plurality of layers.

3. The cellularized nerve regeneration graft of claim 2, wherein the electrospun biodegradable polymer conduit has two layers with one layer comprising aligned biopolymer fibers and the second layer comprising unaligned biopolymer fibers.

4. The cellularized nerve regeneration graft of claim 1, wherein the electrospun biodegradable polymer is a tyrosine-derived or tyrosol-derived polymer.

5. The cellularized nerve regeneration graft claim 1, wherein the fibroblasts form a continuous cell layer on the exterior surface of the conduit at a concentration of about 1.0×10⁵ cells/cm² to about 1.0×10⁶ cells/cm².

6. The cellularized nerve regeneration graft of claim 1, wherein the Schwann cells are substantially evenly distributed throughout the hydrogel matrix or augmented hydrogel matrix within the interior luminal space of the conduit.

7.-8. (canceled)

9. The cellularized nerve regeneration graft of claim 1, wherein the augmented hydrogel matrix comprises a hydrogel selected from the group consisting of: RADA-16 peptides, collagen, gelatin, alginate, hyaluronic acid, or any combination thereof, combined with a growth factor, guidance cue, structural peptide, adhesion factor, other chemical agent, or any combination thereof.

10. The cellularized nerve regeneration graft of claim 1, wherein the system further comprises fibroblasts, other supporting cells, or any combination thereof.

11. The cellularized nerve regeneration graft of claim 10, wherein the concentration of fibroblasts to Schwann cells in the system is about 1:10.

12. A method of making the cellularized nerve regeneration graft of claim 1 comprising:
a. electrospinning a formulation of tyrosine-derived or tyrosol-derived polymer to make the electrospun biodegradable polymer conduit;
b. culturing fibroblasts;
c. seeding the fibroblasts on the exterior surface of the electrospun biodegradable polymer conduit;
d. generating human Schwann cells;
e. embedding the human Schwann cells in the hydrogel matrix or augmented hydrogel matrix to make the system;
f. seeding the system in the interior luminal space of the electrospun biodegradable polymer conduit; and
g. incubating the seeded conduit for about 2 to about 4 weeks to make the cellularized nerve regeneration graft.

13. The method of claim 12, wherein a 3-D printed microdevice is used to facilitate seeding the fibroblasts.

14.-15. (canceled)

16. The method of claim 12, wherein seeding the fibroblasts comprises:
subjecting the electrospun biodegradable polymer conduit to the fibroblasts in a suspension at a concentration of about 1.0×10⁵ cells/mL to about 5.0×10⁵ cells/mL;
rotating the electrospun biodegradable polymer conduit in the fibroblast suspension;
submerging the electrospun biodegradable polymer conduit in a media solution; and
maintaining the submerged electrospun biodegradable conduit for about 10 to about 24 hours until fibroblasts are seeded to the exterior surface of the electrospun biodegradable polymer conduit.

17. The method of claim 12, wherein seeding the system in the interior luminal space of the electrospun biodegradable polymer conduit comprises:
a. capping one end of the electrospun biodegradable polymer conduit;
b. inserting the system into the interior luminal space of the capped conduit to make a filled conduit;
c. submerging the filled conduit in a media solution; and
d. incubating the filled conduit to seed the system and form the cellularized nerve regeneration graft.

18. The method of claim 17, wherein the step of incubating is at about 33 to about 40 degrees C. and about 3% to about 7% CO₂ for about 30 to about 60 minutes, with at least a portion of the media solution being replaced every about 15 to about 30 minutes.

19. The method of claim 18, wherein the step of incubating is further maintained for about 3 weeks with at least a portion of the media solution being replaced every about 1 to about 2 days.

20. A method of repairing an injured peripheral nerve in a patient comprising implanting the cellularized nerve regeneration graft of claim 1 into the patient.

21. The method of claim 20, further comprising adhering the cellularized nerve regeneration graft to a distal end and a proximal end of the injured nerve injury.

22. A cellularized nerve regeneration graft comprising:
a. an electrospun biodegradable polymer conduit having an exterior surface and an interior luminal space;
b. a system filling the interior luminal space of the conduit, the system comprising a hydrogel matrix or augmented hydrogel matrix and Schwann cells; and
c. a plurality of channels running in a longitudinal direction of the electrospun biodegradable polymer conduit and within the system.

23.-32. (canceled)

33. A method of making the cellularized nerve regeneration graft of claim 22 comprising:
a. electrospinning a formulation of tyrosine-derived or tyrosol-derived polymer to make the electrospun biodegradable polymer conduit;
b. generating human Schwann cells;
c. embedding the human Schwann cells in the hydrogel matrix or augmented hydrogel matrix to make the system;
d. culturing resorbable fibers with the human Schwann cells;
e. loading the cultured resorbable fibers into the interior luminal space of the electrospun biodegradable polymer conduit in a longitudinal arrangement and with spacing therebetween;
f. seeding the system into the interior luminal space of the electrospun biodegradable polymer conduit and between the cultured resorbable fibers; and
g. incubating the seeded conduit for about 1 to about 6 weeks, wherein the resorbable fibers dissolve leaving the cellularized nerve regeneration graft having a plurality of longitudinal hollow channels therein.

34. A method of making the cellularized nerve regeneration graft of claim 22 comprising:
a. electrospinning a formulation of tyrosine-derived or tyrosol-derived polymer to make the electrospun biodegradable polymer conduit;
b. generating human Schwann cells;
c. embedding the human Schwann cells in the hydrogel matrix or augmented hydrogel matrix to make the system;
d. loading the resorbable fibers into the interior luminal space of the electrospun biodegradable polymer conduit in a longitudinal arrangement and with spacing therebetween;
e. adding a suspension comprising human Schwann cells into the interior luminal space of the electrospun biodegradable polymer conduit to make cultured resorbable fibers;
f. seeding the system into the interior luminal space of the electrospun biodegradable polymer conduit and between the cultured resorbable fibers; and
g. incubating the seeded conduit for about 1 to about 6 weeks, wherein the resorbable fibers dissolve leaving the cellularized nerve regeneration graft having a plurality of longitudinal hollow channels therein.

35.-38. (canceled)
